(19) United States
(12) Patent Application Publication
(10) Pub. No.: US 2018/0192912 A1
(43) Pub. Date: Jul. 12, 2018

(54) SYSTEM AND METHOD FOR DETERMINING CALORIMETRIC PERFORMANCE AND REQUIREMENTS

(71) Applicant: TreyMed, Inc., Sussex, WI (US)

(72) Inventors: Robert H. Ricciardelli, Waukesha, WI (US); Michael J. Marking, Menomonee Falls, WI (US)

(21) Appl. No.: 15/868,406

(22) Filed: Jan. 11, 2018

Related U.S. Application Data
(60) Provisional application No. 62/445,012, filed on Jan. 11, 2017.

(51) Int. Cl. A61B 5/083; A61B 5/087; A61B 5/00; A61B 5/097

(52) U.S. Cl. CPC A61B 5/083; A61B 5/087; A61B 5/0004; A61B 5/0836; A61B 5/097; A61B 5/0833; A61B 5/742

(57) ABSTRACT

A calorimetric performance monitoring system includes an analyzer that is fluidly connected to an in-stream respiration flow sensor. The system includes a controller that is configured to determine a flow rate of respiration flow and at least a portion of a composition of the respiration flow on a breath-by-breath basis and temporally associate the respiration flow value and the determined portion(s) of the composition of the respiration flow and segregate non-steady state respiration performance data from steady state respiration performance data and determine a calorimetric performance from the steady state respiration performance data.

### CROSS REFERENCE TO RELATED APPLICATIONS

This application claims priority to U.S. Provisional Patent Application Ser. No. 62/445,012 filed on Jan. 11, 2017 titled “System and Method for Determining Calorimetric Performance and Requirements” and the disclosure of which is incorporated herein.

### FIELD OF THE INVENTION

The present application relates to a system and method for monitoring respiration performance and, more particularly, to a respiration monitoring system that is configured to monitor respiratory and physiological performance of a person being monitored and which is configured to determine the calorimetric performance and/or requirements of a patient from information associated with the patient respiration performance. The invention provides a system and method for real time, breath-by-breath side-stream monitoring of a patient. The system monitors respiration flow rate and flow constituents to assess various parameters of a patient's physiological condition, respiration performance, and metabolic calorimetric performance.

### BACKGROUND OF THE INVENTION

As disclosed in Applicant's related U.S. Pat. Nos. 8,459,261 and 6,659,962; it is generally well accepted that monitoring respiration performance provides diagnostic insight into a patient's overall health as well as specific respiratory function. Applicant's currently pending U.S. patent application Ser. No. 15/195,184 expands upon the disclosures of the '261 and '962 patents and discloses a system and method of temporally aligning the flow and constituency data acquired during patient breath cycles.

Indirect calorimetry is a measurement technique that is used to assess and evaluate a subject's metabolic state. The system and assembly disclosed in the patent documents cited above has been further improved to make indirect calorimetric measurements of a subject by non-invasively measuring inspired and expired gases at the mouth of a subject. As disclosed in the patent documents cited above, the various measured and/or determined respiration performance parameters include assessments at least the concentrations of gases such as carbon dioxide (CO2), oxygen (O2), as well as patient respiration flow volumes and durations. The respiration performance monitoring systems disclosed therein are configured to integrate or otherwise correlate flow constituent concentration values with aligned flow values as well as gas flow volumes.

Although the tidal respiration flow compositions and volumes as disclosed in applicants U.S. Pat. Nos. 8,459,261 and 6,659,962 provide information that can be utilized to assess a patient's respiration performance, such information is indicative of only a portion of a patients underlying overall health or physiologic condition or performance. Those skilled in the art will appreciate that the respiration performance data acquired by respiration performance monitoring systems or devices such as those disclosed in applicants related patent documents, as well as other similar systems, lack any consideration or capability of assessing a patients physiologic calorimetric performance.

Commonly, extraneous patient calorimetric information is discretely acquired and maintained. Unfortunately, such approaches suffer from various shortcomings. Such an approach lacks any consideration of a patient's respiration performance data that could be considered contemporaneous relative to the calorimetric information so as to attain a more robust representation of a patient's overall health condition or assessment. Such approaches are also susceptible to a plethora of errors including human error associated with inaccurate assessment and/or recordal of patient nutritional inputs or the like and/or inaccurate, incomplete, or nonexistent association between the discrete patient calorimetric information with respect to patient respiration performance.

Such approaches also commonly present a substantial delay between a discrete determination of a patient's calorimetric performance and discrete respiration performance that they are rendered incapable of producing a temporally or time-wise aligned indication as to a patient's respiration and calorimetric performance. Such approaches exacerbate the potential for incorrect diagnosis and/or information associated with a patient's physiologic condition and negates consideration of a patient's contemporaneous respiration performance on the determination of the patient's calorimetric performance. Such approaches are also commonly labor intensive and patient intrusive and present substantial lags in the availability of a patient's calorimetric performance data relative to real-time respiration performance.

Accordingly, there is a need for a system and method for more readily determining a patient's current calorimetric performance. More preferably, there is a need for a system and method of determining patient calorimetric performance information from a patient's respiration performance. There is a further need of a system and method for determining a patient's calorimetric performance that is less intrusive, preferably non-invasive, and labor or clinician/patient interaction intensive. There is a further need for a system and method for determining a patient's physiologic performance that can more readily determine and align patient respiration and calorimetric performance to provide a more contemporaneous determination of a patient's overall physiologic condition.

### BRIEF DESCRIPTION OF THE INVENTION

The present application is directed to a calorimetric performance monitoring system that overcomes one or more of the aforementioned drawbacks. One aspect of the application discloses a calorimetric performance monitoring system that includes an analyzer that is configured to be fluidly connected to a flow sensor that is constructed to be disposed in a respiration flow path. A controller is associated with the analyzer and configured to determine a respiration flow and a composition of at least a portion of the respiration flow and to segregate the acquired respiration flow and composition data between steady state respiration performance data and non-steady state respiration performance data and determine a value associated with calorimetric performance of a subject based on the steady state respiration performance data.

Another aspect of the present application that is usable or combinable with one or more of the above aspects discloses a method of forming a calorimetric performance monitoring system. The method includes providing a flow sensor that is constructed to be disposed in a respiration flow stream and which includes at least a first, a second, and a third port formed through a sidewall of the flow sensor. An analyzer is provided that is constructed to be fluidly connected to the first port, the second port, and the third port of the flow sensor. The method includes providing a controller that is configured to control operation of the analyzer and to determine a flow value through the flow sensor from information associated with the first and the second ports of the flow sensor and determine a flow composition value associated with a respiration flow stream from a sample of the respiration flow stream communicated to the analyzer via the third port. The controller is further configured to cause the analyzer to generate an alignment signal that is communicated to the flow sensor via one of the first, the second, and the third ports and temporally align the flow value and the composition value from information returned to the analyzer attributable to the alignment signal.

Another aspect of the present application that is usable or combinable with the above aspects discloses a method of determining calorimetric performance from respiration performance data. The method includes determining a flow and at least a portion of a composition of a respiration flow and determining a calorimetric performance of a user from data associated with the determined flow and at least a portion of the composition of the respiration flow. Determination of the calorimetric performance includes segregation of the respiration performance data and omission of at least a portion of acquired data that is attributable to non-steady respiration performance.

These and various other features, aspects, and advantages of the present invention will be made apparent from the following detailed description and the drawings.

### BRIEF DESCRIPTION OF THE DRAWINGS

The drawings illustrate one preferred embodiment presently contemplated for carrying out the invention.

FIG. 1 is a graphical representation of a calorimetric performance respiration performance monitoring system configured in accordance with the present invention;

FIG. 2 is a graphical representation of an analyzer of the monitoring system shown in FIG. 1;

FIG. 3 are exemplary displays of respiration performance monitoring data acquired with the monitoring system shown in FIG. 1; and

FIG. 4 is a graphical representation of a display of the calorimetric and respiration performance monitoring information generated from the information acquired from the monitoring system shown in FIG. 2 and determined by the analyzer shown in FIG. 2.

### DETAILED DESCRIPTION OF THE PREFERRED EMBODIMENT

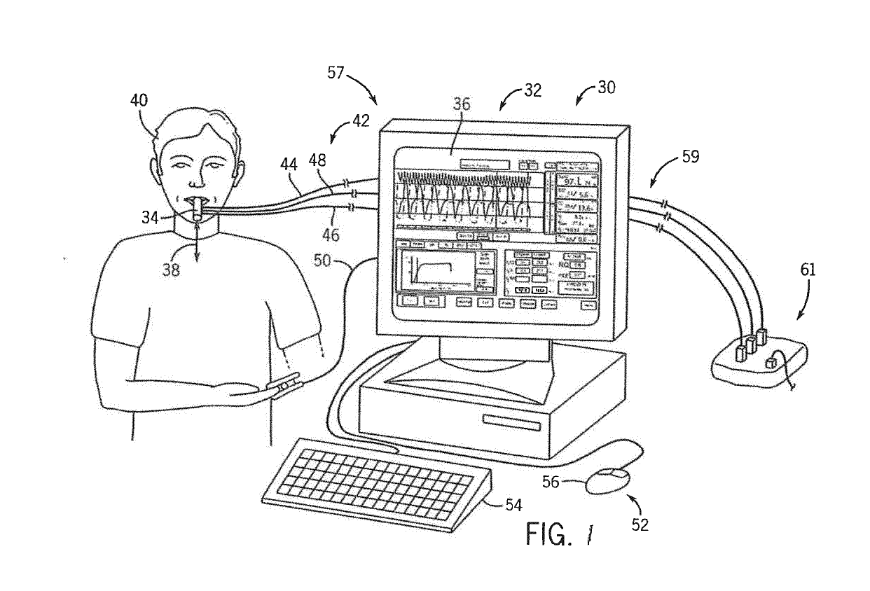

As shown in FIG. 1, the present invention is directed to a monitoring system 30 that includes a control or analyzer 32, a sensor 34, and a display 36 and is configured to determine a calorimetric performance and/or need of a user associated with sensor 34. Sensor 34 is constructed to engage a respiration flow, indicated by arrow 38, associated with a participant or patient 40. A number of lumens or tubes 42 operatively connect sensor 34 to analyzer 32. A first tube and a second tube 44, 46 are connected to sensor 34 which is operative to detect a pressure differential of respiration flow 38 in sensor 34. The pressure differential associated with tubes 44, 46 is utilized by analyzer 32 to calculate a respiration flow value with respect to sensor 34. A third tube 48 acquires an aspirated sample of respiration flow 38 and communicates the sample to analyzer 32. A physiological detector, preferably a heart rate monitor 50, is also connected to analyzer 32 and constructed to communicate a patient cardiac status to analyzer 32. Preferably, monitor 50 is configured to monitor both the pulsatile effects of the patient's cardiac cycle as well as the saturated oxygen content of the patient's circulation system. Applicant's issued and currently pending patent documents U.S. Pat. Nos. 8,459,261; 6,659,962; and U.S. Patent Application Publication No. 2017/0367618 further describe the construction and operation of a monitoring system 30 useable with the present invention and the disclosures of which are incorporated herein.

Analyzer 32, having acquired the data or signals from tubes 42 and heart rate monitor 50, generates time aligned and composition corrected respiration information and outputs the information at display 36 as explained further below. Analyzer 32 includes optional user inputs 52 that allow a user to selectively configure the operation of analyzer 32 and the output of display 36 such that analyzer 32 and display 36 generate and output the desired information, respectively. As disclosed further below, it is further appreciated that display 36 can be constructed as a touch screen and/or personally portable display such that a user or technician can manipulate the display results thereof and operation of analyzer 32 by touching selected areas of the display without utilization of auxiliary input devices such as a keyboard 54 and/or a mouse 56.

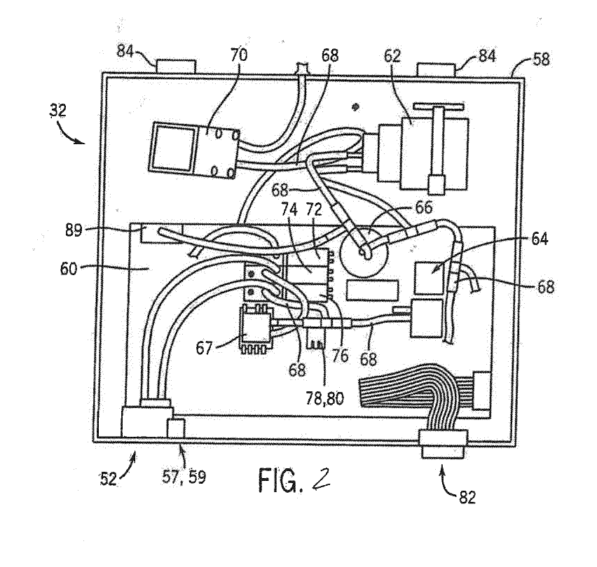

Supported by an understanding associated with the operation of system 30 disclosed in the patent documents cited above, and as described further with respect to FIG. 2, analyzer 32 includes a first input 57 and a second input 59 to allow multiple gas sources to concurrently be connected to analyzer 32. As shown in FIG. 2, first input 57 is connected to sensor 34 and second input 59 is connected to another sensor, a Douglas bag, gas cylinder, or container 61. It is appreciated that container 61 can be configured to contain a volume of a known gas or a volume of a gas collected from another patient. Such a configuration allows monitoring system 30 to monitor and assess multiple gas sources. Such a configuration is particularly useful in environments where monitoring of several patients is desired or where patients with reduced respiration tidal volumes, such as premature babies, have such low respiration tidal volumes that in-line collection of a portion of the respiration sample is required to assess the composition of the respiration gases.

Referring to FIG. 2, analyzer 32 includes a housing 58 having a control or controller 60 contained therein. An oxygen sensor 62, a nitrous oxide sensor 64, and a carbon dioxide sensor 66, and a flow sensor 67 are also positioned in housing 58. It is understood that oxygen sensor 62 may be operable under any of a number of technology based methodologies such as laser, acoustic, solid state, amperometric such as galvanic, or potentiometric. A number of tubes 68 interconnect sensors 62, 64, 66 and communicate respective portions of the acquired flow through the analyzer. A pump 70 and a number of valves 72, 74, 76 control the directional passage of the respiration flow sample through analyzer 32. Analyzer 32 includes a humidity sensor 78 and a temperature sensor 80 configured to monitor both ambient temperature and humidity as well as temperature and humidity of the respiration flow. It is further appreciated that analyzer 32 may include an optional heater and/or humidifier to communicate thermal energy and/or moisture to a patient via the respiration flow cycle. It is further appreciated that analyzer 32 may also be configured to administer medications via the respiration cycle where necessary or desired.

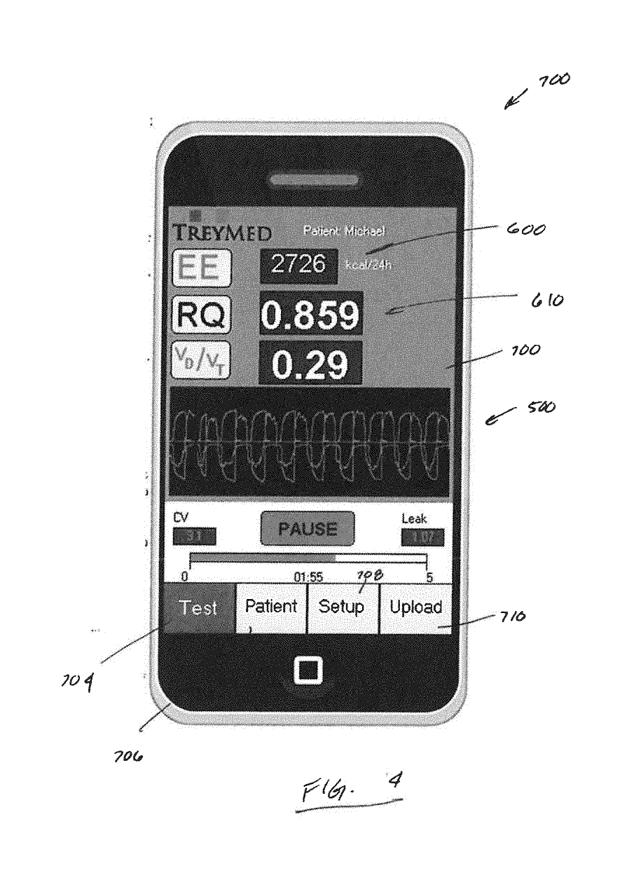

First input 57 and second input 59 extend through housing 58 and are constructed to removably engage the tubes 42 connected to sensor 34 or container 61 as shown in FIG. 1. An electrical connector 84 also extends through housing 58 and is constructed to communicate information generated by analyzer 32 to external devices such as personal computers, personal data assists (PDA's), cell phones, or the like. Alternatively, it is further understood that analyzer 32 may include a wireless interface to allow wireless communication of the information acquired and calculated by analyzer 32 to external devices such as a cell phone or like as discussed further below with respect to FIG. 4. Analyzer 32 includes an input connector 82 constructed to communicate information from patient monitor 50 to the analyzer. Input 84 is constructed to removably connect monitor 50 to analyzer 32 to communicate the information acquired by monitor 50 to the analyzer 32. It is understood that inputs and connectors 84 may be any conventional connection protocol such as serial pin connectors, USB connectors, or the like, or have a unique configuration. Analyzer 32 further includes a leak test valve 89, the operation of which is described further below with respect to the automatic calibration and performance monitoring of analyzer 32. It is appreciated that the relatively compact and lightweight nature of analyzer 32 provides a respiration monitoring system 10 that is highly portable and operable with a number of sensors. The determination and alignment of the respiration flow and composition information is further described in applicant's related patent documents cited above.

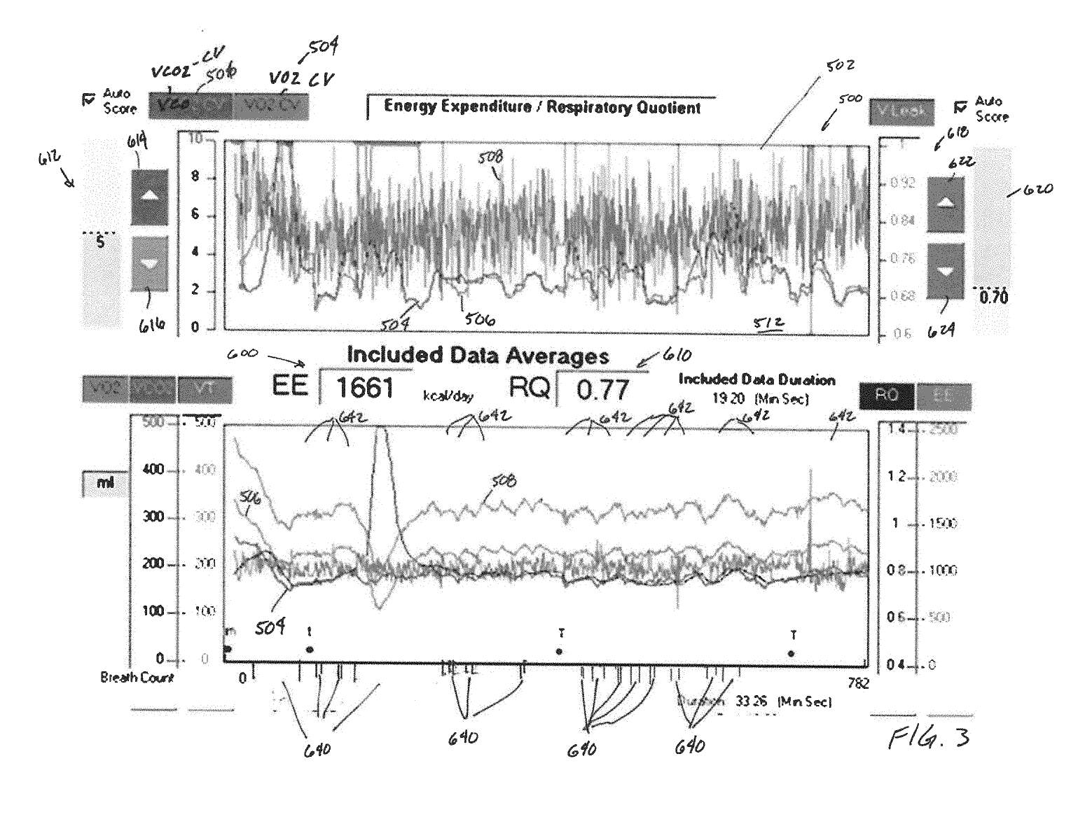

FIG. 3 shows an exemplary time-aligned respiration output trend or output 500 generated by analyzer 32 as well as a calorimetric performance value 600 determined during use thereof. Output 500 includes a trend window 502 that is configured to display a volumetric coefficient of variation adjusted carbon dioxide value 504, and a volumetric coefficient of variation adjusted oxygen values 506, and a ventilator leak ratio (difference between inspired and expired patient flow) 508 in a common screen 512. The generation of the various other flow, concentration, and alignment trend lines is further disclosed in applicants related U.S. Patent Application Publication No. 2017/0367618 and U.S. Pat. Nos. 8,459,261; 6,659,962 and the disclosures of which are incorporated herein.

As disclosed therein, each of the respiration cycle concentration values are temporally aligned along the data trend. The carbon dioxide concentration and the oxygen concentration values are generally produced as mirror images of one another such that quick viewing and interpretation of the breath data can be achieved. It is further appreciated that the oxygen concentration data is acquired by scaling the respiration data by a factor such that it correlates to the carbon dioxide concentration value. Alternatively, it is understood that analyzer 32 may be configured to monitor the oxygen content deficiency and that this value may then be inverted to generally mimic the carbon dioxide concentration value. Both configurations provide a carbon dioxide and oxygen concentration displayed value that is readily assessable.

Analyzer 32 and controller 60 associated therewith are configured to indirectly determine a calorimetric value from information associated with the respiration performance acquired from sensor 32 to determine a person's nutritional performance or need from the respiration flow and concentration information acquired and/or determined by analyzer 32. The determined volumetric carbon dioxide 506 (VCO2) and volumetric oxygen 504 (VO2), are used to calculate a Respiratory Exchange Ratio (RER) and calorimetric performance value 600 as Energy Expenditure (EE).

The Respiratory Exchange Ratio is the ratio of volumetric carbon dioxide 506 divided by volumetric oxygen 504. The respiratory quotient (RQ) 610 is this same ratio of volumetric carbon dioxide 506 divided by volumetric oxygen 504 but adjusted to reflect its occurrence at a cellular level. Respiratory Exchange Ratio will generally equal the respiratory quotient when the subject is at steady state, such as when at rest or during prolonged, continuous constant activity. These Respiratory Exchange Ratio and respiratory quotients are not the same when respiration is in a dynamic or non-steady state condition such that, determining a calorimetric value from information associated with the respiration performance information associated with sensor 32 must first determine and segregate the steady state respiration performance data and the non-steady state respiration performance data. Understandably, the non-steady state respiration performance data cannot be wholly ignored as the same is necessary to assess the overall respiration performance information. Accordingly, to determine the calorimetric performance of the user associated with sensor 32 requires selective segregation of the non-steady state and steady state respiration performance data.

Resting Energy Expenditure (REE), and equivalently Resting Metabolic Rate (RMR) is the measured Energy Expenditure when a subject in a resting state. The values associated with the Resting Energy Expenditure and respiratory quotient are used for nutritional assessment or the determination of a subject's calorimetric performance or need. Determination of these values when the subject is not in a resting state detracts from the accuracy associated with the determination of the Resting Energy Expenditure and Resting Metabolic Rate and thereby detracts from the accuracy associated with the subject's calorimetric requirements and/or performance.

System 32 includes accommodation of a Coefficient of Variation (CV) 612 associated with determining stability of the respiration performance. Typically, clinicians rely on the “Coefficient of Variation” (CV) of the data to be low, typically less than between 5% to 10%, for some period of “time”. Both “CV” level and “time” requirements depend typically on institutional guidelines as determined by industry (professional journals, academic) guidance. The value associated with CV 612 is adjustable 614, 616 to accommodate different institutional requirements and/or operator preferences.

System 32 and the controller 60 associated therewith automatically collects and segregates respiration performance data for use in determining a subject's calorimetric performance and/or requirement. The operator (or institution) may configure the software associated with operation of controller 60 to search for respiration performance stability based on multiple criteria such as the coefficient of variation associated with each of carbon dioxide and oxygen volumes and/or concentrations, an adjustable ventilator leak accommodation 618, 620, 622, 624 (ratio of expired divided by inspired tidal volume), etc. System 32 simplifies the collection process associated with suitable steady state performance data. That is, system 32 determines whether a suitable degree of respiration performance stability is attained and whether a required sample time duration is reached in a manner that shortens the overall collection time associated with acquiring sufficient data to determine a calorimetric performance or need associated with the subject.

As shown in the lower graphic portion of FIG. 3, during respiration performance monitoring, various portions of the data 640 associated with the respiration performance information will be unsuitable, or have a non-steady state respiration performance characteristic, that renders then unsuitable for inclusion in the determination of the calorimetric performance value. System 32 disregards or omits contribution of data 640 in the determination of the calorimetric performance value such that preferably only steady state respiration performance data 642 contributes to the determination of the calorimetric performance value. Such a configuration mitigates the detriments associated with oversampling or contribution of non-steady state respiration performance data to the determination of the calorimetric value common to other systems and does so in a nearer real-time basis relative to acquisition of the respiration performance information associated with determining the calorimetric performance of a subject.

In another aspect of the invention, as shown in FIG. 4, system 32 is configured to communicate with personal electronic devices 700, such as cell phones of the like, either wirelessly or via a suitable wired connection, to communicate the information acquired and/or determined thereby, such as energy expenditure 600, respiratory quotient 610, and/or other data information such as selected trends 500 of the like. Devices 700 include a dashboard 702 configured to provide the desired information and preferably one or more inputs, 704, 706, 708, 710 associated with configuring dashboard 702 and/or the information associated therewith, to a user. Preferably, device 700 is configured to allow retention of the information assessed thereat, includes the calorimetric performance information associated with the value of energy expenditure 600 to allow a clinician to adjust a subjects treatment to maintain a desired calorimetric balance.

Therefore, one aspect of the present application includes a calorimetric performance monitoring system having an analyzer that is configured to be fluidly connected to a flow sensor that is constructed to be disposed in a respiration flow path. A controller is associated with the analyzer and configured to determine a respiration flow volume and a composition of at least a portion of the respiration flow. The controller is further configured to segregate acquired respiration flow data between steady state respiration performance data and non-steady state respiration performance data and determine a value associated with a calorimetric performance of a subject associated with the flow sensor based on the steady state respiration performance data.

Another aspect of the present application that is usable or combinable with one or more of the above features or aspects discloses a method of forming a calorimetric performance monitoring system. The method includes providing a flow sensor that is constructed to be disposed in a respiration flow stream and which includes at least a first, a second, and a third port formed through a sidewall of the flow sensor. An analyzer is provided that is constructed to be fluidly connected to the first port, the second port, and the third port of the flow sensor. The method includes providing a controller that is configured to control operation of the analyzer and determine a flow value through the flow sensor from information associated with the first and the second ports of the flow sensor and determine a flow composition value associated with a respiration flow stream from a sample of the respiration flow stream communicated to the analyzer via the third port. The controller is further configured to cause the analyzer to generate an alignment signal that is communicated to the flow sensor via one of the first, the second, and the third ports and temporally align the flow value and the composition value from information returned to the analyzer attributable to the alignment signal and determine a calorimetric performance associated with the source of the respiration flow stream.

Another aspect of the present application that is combinable or useable with one or more of the above aspects discloses a method of determining calorimetric performance from respiration performance data. The method includes determining a flow and at least a portion of a composition of a respiration flow stream and determining a calorimetric performance from data associated with the determined flow and at least a portion of the composition of the respiration flow that includes removing at least a portion of non-steady state respiration performance data from the determination of the calorimetric performance.

It is to be understood that specific details described above are not to be interpreted as limiting the scope of the invention, but are provided merely as a basis for teaching one skilled in the art to variously practice the present invention in any appropriate manner. Changes may be made in the details of the various methods and features described herein, without departing from the spirit of the invention

What is claimed is:

1. A calorimetric performance monitoring system, the system comprising:
an analyzer configured to be fluidly connected to a flow sensor that is constructed to be disposed in a respiration flow path; and
a controller associated with the analyzer and configured to determine a respiration flow volume and a composition of at least a portion of the respiration flow, the controller being further configured to segregate acquired data between steady state respiration performance data and non-steady state respiration performance data and determine a value associated with a calorimetric performance of a subject associated with the flow sensor and based on the steady state respiration performance data.

2. The system of claim 1 wherein the controller is further configured to allow a user to set at least one threshold associated with determining segregation between the steady state and non-steady state respiration performance data.

3. The system of claim 2 wherein the controller is further configured to allow a user to set a second threshold such that the first and second thresholds must each be satisfied for respiration performance data to qualify as steady state performance data associated with determining the value associated with the calorimetric performance.

4. The system of claim 1 wherein the controller is configured to exclude non-steady state respiration performance data associated with each breath cycle during determination of the value associated with the calorimetric performance.

5. The system of claim 1 wherein the controller is further configured to generate an alignment signal that is communicated to the flow sensor and a portion of which is therefrom returned to the analyzer and the controller utilizes the alignment signal to temporally align acquired respiration flow data and composition data in response to information associated with the alignment signal.

6. The system of claim 1 wherein the flow sensor includes a first and a second port that are connected to the analyzer and associated with determining a flow through the sensor and a third port that communicates a sample of the flow to the analyzer.

7. A method of forming a calorimetric performance monitoring system comprising:
providing a flow sensor that is constructed to be disposed in a respiration flow stream and which includes at least a first, a second, and a third port formed through a sidewall of the flow sensor;
providing an analyzer constructed to be fluidly connected to the first port, the second port, and the third port of the flow sensor; and
providing a controller configured to control operation of the analyzer and determine a flow value through the flow sensor from information associated with the first and the second ports of the flow sensor and determine a flow composition value associated with a respiration flow stream from a sample of the respiration flow stream communicated to the analyzer via the third port, the controller being further configured to cause the analyzer to generate an alignment signal that is communicated to the flow sensor via one of the first, the second, and the third ports and temporally align the flow value and the composition value from information returned to the analyzer attributable to the alignment signal.

8. The method of claim 7 wherein the controller is further configured to segregate acquired flow data and composition data between steady state respiration performance data and non-steady state respiration performance data and determine a value associated with a calorimetric performance based on the steady state respiration performance data.

9. The method of claim 8 wherein the controller is further configured to characterize the flow data and composition data that includes a contribution attributable to the alignment signal as non-steady state respiration performance data.

10. The method of claim 8 further comprising providing a display configured to generate a visual output of the value associated with the calorimetric performance.

11. The method of claim 10 furthering comprising providing a wireless communication between the analyzer and the display.

12. The method of claim 8 further comprising configuring the display to concurrently output the value associated with the caloric performance, the flow value, and the flow composition value wherein each of the value associated with the caloric performance, the flow value, and the flow composition value a respiration flow stream are temporally aligned with one another relative to a discrete portion of the respiration flow stream.

13. A method of determining calorimetric performance from respiration performance data, the method comprising:
determining a flow and at least a portion of a composition of a respiration flow stream; and
determining a calorimetric performance from data associated with the determined flow and at least a portion of the composition of the respiration flow that includes removing at least a portion of non-steady state respiration performance data from the determination of the calorimetric performance.

14. The method of claim 13 further comprising segregating the determined flow and the determined portion of the composition of the respiration flow into steady state respiration performance data and the non-steady state respiration performance data.

15. The method of claim 13 further comprising communicating a sample of the respiration flow stream from an in-stream flow sensor and an analyzer.

16. The method of claim 15 further comprising communicating an alignment signal from the analyzer to the flow sensor and acquiring data with the analyzer that is attributable to the alignment signal and aligning the determined flow and the determined composition in a timewise manner as a function of operation of the alignment signal.

17. The method of claim 13 further comprising displaying a value associated with the determined calorimetric performance concurrently with the determined flow and composition associated with the respiration flow stream wherein at least one of the values associated with the calorimetric performance and the determined flow and determined composition have been shifted in a time domain to be aligned with one another.
